(19) United States
(12) Patent Application Publication
(10) Pub. No.: US 2008/0108702 A1
(43) Pub. Date: May 8, 2008

(54) Use of Acetyl-Leucine for Preparing a Drug for Treating Balance Disorders

(76) Inventor: Christophe Przybylski, Villefranche De Lauragais (FR)

Correspondence Address: BIRCH STEWART KOLASCH & BIRCH, PO BOX 747, FALLS CHURCH, VA, 22040-0747, US

(21) Appl. No.: 11/886,419

(22) PCT Filed: Mar. 17, 2006
(86) PCT No.: PCT/EP06/60841

§ 371 Date: Sep. 17, 2007

(30) Foreign Application Priority Data
Mar. 18, 2005 (FR) 0502696

(51) Int. Cl. A61K 31/198; A61P 27/16

(52) U.S. Cl. 514/561

(57) ABSTRACT

The invention concerns the use of acetyl-leucine for preparing a drug for treating acute deafferentiation in patients whose vestibular deficit is less than (88%), advantageously less than (75%) to caloric tests.

The present invention relates to the use of acetyl-leucine for the preparation of a medicament for the treatment of balance disorders.

Acetyl-leucine in racemate form and the salts of same are known for their effectiveness in the treatment of vertigo of various origins, notably Meniere's vertigo and vertigo of inflammatory (vestibular neuritis) or toxic origin.

Acetyl-leucine is marketed by Pierre Fabre Medicament in racemate form as an anti-vertigo medicament under the name Tanganil®. Clinical results relating to said medicament reported by various authors demonstrate an improvement in vertigo symptomatology in more than 95% of cases, including the disappearance of vertigo attacks.

Acetyl-leucine is also known to accelerate vestibular compensation after unilateral labyrinthectomy in the guinea pig, whereas it has no effect on normal vestibular functioning (P. P. Vidal et al., Eur. J. Neurosci. (2001), 13(4), 735-748).

Recent studies have shown that acetyl-leucine administered by oral route at a dose of 28 mg per kg per day facilitates the recovery of locomotor equilibrium in cats having undergone vestibular neurotomy (Sun Jiurong, Zhongguo Yingyong Shenlixue Zasti Bianjibu (1997), 13(3), 257-260).

Vertigo attacks are related to disequilibrium in the membrane potentials of median vestibular nuclei neurons. This disturbance of the system is expressed by neuronal hyperpolarization or depolarization. One treatment for vertigo attacks thus consists of attenuating the imbalance by returning the membrane potentials of these neurons to their resting potential.

Vestibular neurotomy is a surgical technique for the treatment of vertigo cases that are incapacitating and highly resistant to conservative therapies. The principle involves deafferentation of the peripheral vestibular system by sectioning the vestibular nerve while maintaining the integrity of the cochlear and facial nerves.

Following a unilateral vestibular neurotomy, patients often experience compensation which within a week relieves the postural or oculomotor static deficits observed during the acute stage. On the other hand, dynamic deficits also recover but never fully (C. De Waele, review ref. AN 19824, 33rd Symposium of the International Society of Otoneurology, Geneva, May 14-15, 1999).

However, such compensation is observed only when neurotomy is performed at an early stage of a tumor's development; when the tumor is diagnosed late, the transmission of vestibular information has long stopped by the time neurotomy is performed.

Surprisingly, however, the inventors have shown that acetyl-leucine, which has no effect in a patient population exhibiting no vestibular functioning before neurotomy, accelerates the compensation observed in a population of patients exhibiting residual vestibular functioning.

Consequently, the present invention relates to the use of acetyl-leucine for the preparation of a medicament intended to treat acute deafferentiation syndromes in patients whose vestibular deficit is less than 88%, advantageously less than 75%, to caloric tests.

In one advantageous embodiment of the invention, said syndromes are intermittent acute deafferentiation syndromes resulting from an imbalance in the predominantly unilateral transmission of sensory impulses in the auditory nerve.

In another advantageous embodiment of the invention, the syndromes are intermittent acute deafferentiation syndromes resulting from any event causing a unilateral loss of sensory impulse transmission in the auditory nerve, accompanied by vertigo.

Acute deafferentiation syndromes may result from surgical unilateral neurotomy. Intermittent acute deafferentiation syndromes may result in particular from unilateral neurotomy selected from the group comprising traumatic unilateral neurotomy, unilateral neurotomy related to acute ischemia of the auditory nerve, unilateral neurotomy due to extrinsic compression, unilateral neurotomy due to structural edemas and unilateral neurotomy resulting from endogenous viral attacks.

Vestibular deficit is evaluated by caloric testing which provides information about the vestibular reflex, i.e., the capacity of the vestibule to respond to stimulation (irrigation of the external auditory meatus by warm water followed by cold).

In the sense of the present invention, acetyl-leucine means (DL)-acetyl-leucine, (L)-acetyl-leucine, (D)-acetyl-leucine and pharmaceutically acceptable salts of same.

In the context of the present invention, acetyl-leucine may be administered by oral route at a dose between 500 mg and 10 g per day, advantageously between 1 g and 2 g per day.

The inventive acetyl-leucine may also be administered by intravenous route at a dose between 500 mg and 1 g per day, without interruption.

The inventive acetyl-leucine may be provided in any dosage form suitable for oral administration, notably in the form of granules, powders, hard capsules, soft capsules, gelatin capsules, lyophilized tablets, syrups, emulsions, suspensions or solutions, or in any form suitable for intravenous administration.

The following examples illustrate the invention.

### EXAMPLE 1

### Effect of Tanganil® on the Ability to Maintain a Standing Position Eight Days Following Neurotomy

1.1. Protocol

1.1.1. Subject Selection and Treatment

Sixty patients with an indication for acoustic neurinoma excision or for vestibular neurotomy were included and randomized. Patients were treated for 60 days either with Tanganil® (six injections of two 500 mg vials by IV route, with an interval of 12 hours between injections, the first taking place one hour after the surgery, followed by an oral treatment at a dose of two 500 mg tablets, one in the morning and one in the evening) or with a placebo.

1.1.2. Measurement of Vestibular Deficit

Vestibular deficit was measured according to the methods described in Vertigo and the practitioner [Les vertiges et le praticien] by P. Tran Ba Huy and C. De Waele (Coll Pathologie Sciences Formation [in French], Ed. John Libbey Eurotext, Paris, 1996) and in Vertigo [Les vertiges] by G. Rancurel, O. Sterkers, E. Vitte, foreword by G. Freyss (Ed. Méd. Spécia. [in French], 1989).

Vestibular reflex was tested at day zero (D₀) before neurotomy by caloric testing to objectivize vestibular hyporeflexia or areflexia. This test consists of successively irrigating with warm water then cold the right external auditory meatus then the left, causing in the semicircular canal an endolymph current which moves the ampullary crest. Caloric response is expressed by the appearance of a nystagmus that beats toward the side of the stimulated ear when warm water is applied and toward the opposite side when cold water is applied. The nystagmic response was recorded by video nystagmography and its frequency calculated, i.e., the number of nystagmic beats occurring between the 60th and 90th seconds following the initiation of stimulation. This number quantifies the vestibular response.

The difference in responses between one ear and the other expresses so-called hypovalence, i.e., the difference between the warm and cold responses on the left side and the warm and cold responses on the right side, divided by the total number of responses. In normal subjects, hypovalence is less than 15%. Hypovalence is the main sign of damage to the peripheral vestibular apparatus.

Results from 56 subjects were analyzed: 25 patients had a vestibular deficit greater than 75% and 31 patients had a vestibular deficit less than or equal to 75%.

1.1.3. Measurement of Standing Position at D₈

The principle criterion was “able to maintain a standing position at D₈” (Yes/No)

1.2. Results

The principle analysis (of 49 patients with no major deviation regarding evaluation of the principle criterion at D₈) showed no statistically significant difference between Tanganil® (13 of 22 patients, or 59.1%, able to maintain a standing position at D₈) and the placebo (12 of 27 patients, or 44.4%, able to maintain a standing position at D₈).

Similarly, among the 25 patients whose vestibular deficit was greater than 75%, no significant difference between the two treatment groups was demonstrated.

On the other hand, among 31 patients whose vestibular deficit was less than or equal to 75%, a statistically significant difference was observed between the two treatment groups in favor of Tanganil® (χ²=0.048, less than 5%). In this subgroup, 12 of 16 patients given Tanganil® (75%) were able to maintain a standing position compared to 6 of 15 patients given placebo (40%).

### EXAMPLE 2

### Effect of Tanganil® on Various Symptoms Evaluated Using Visual Analog Scales (VAS)

2.1. Protocol

Subjective signs (vertigo and vomiting) were rated by patients on a 10 cm VAS from “absent” to “highly incapacitating”.

2.2. Results

2.2.1. Effects on Vertigo

In the subgroup of patients whose vestibular deficit was less than or equal to 75%, a statistically significant difference at D₈ between the group treated with Tanganil® and the group treated with placebo was observed (Wilcoxon test, p=0.037, less than 5%).

An average decrease of −23.5 mm on the vertigo VAS among patients treated with Tanganil® was observed compared to an average increase of +4.7 mm for placebo.

On the other hand, no significant difference was observed in the subgroup of patients whose vestibular deficit was greater than 75% (Wilcoxon test, p=0.154, greater than 5%).

2.2.2. Effects on Vomiting

No significant difference was demonstrated in the subgroup of patients whose vestibular deficit was greater than 75%. On the other hand, for the subgroup of patients whose vestibular deficit was less than or equal to 75%, a significant difference between the two groups appeared, namely a reduction in vomiting severity for patients given Tanganil®.

### EXAMPLE 3

### Effect of Tanganil® on Balance, Evaluated by Static Posturography

Postural tonus is expressed by the human body's overall bearing, setting the various joints in specific positions interdependently, primarily in response to gravitational forces. Vestibular afferents, extrinsic ocular musculature, proprioceptive afferents and visual afferents play a major role in maintaining a standing position. Subjects are never perfectly motionless when standing at rest: they are constantly oscillating.

3.1. Protocol

Static posturography (not performed at D₈ if the subject could not remain standing) was carried out in a standing position under four conditions (eyes open or closed, with or without foam carpeting). With foam carpeting and/or eyes closed, patients are placed under conditions which minimize the role of visual and proprioceptive information.

Shifts in a subject's center of gravity during a given time period are recorded in a statokinesigram. This plot makes it possible to study oscillation amplitudes and surface areas. “Anteroposterior” oscillations can be separated from “right-left” oscillations.

3.2. Results

In the subgroup of patients whose vestibular deficit was greater than 75% at D₈, regardless of which protocol was used (eyes open or closed and/or with or without foam), either no significant difference was observed between Tanganil® and the placebo or a significant difference was observed between the two groups in favor of the placebo.

On the other hand, regardless of experimental conditions a significant difference between treatment groups in favor of Tanganil® was demonstrated only in the subgroup of patients whose vestibular deficit was less than or equal to 75%.

Moreover, regardless of the experimental conditions under which it was demonstrated, a significant reduction between the treatment groups in favor of Tanganil® was demonstrated only on “right-left” postural oscillations.

With regard to right/left posturography, no significant difference was observed between Tanganil® and placebo at D₈ for the “without foam, eyes open” test. On the other hand, at D₈, in the group of patients whose vestibular deficit was less than or equal to 75%, there was a significant difference (p=0.034) between the two treatments in favor of Tanganil® (+7.9 mm vs. +19.9 mm) in the “with foam, eyes open” test.

In this same group of patients, there was also a significant difference between the two treatments in favor of Tanganil® in the “without foam, eyes closed” test (+2.8 mm vs. +10.9 mm; p=0.028) and in the “with foam, eyes closed” test (+5.4 mm vs. +13.5 mm; p=0.016).

The results thus obtained demonstrate the positive action of Tanganil® on the reduction of vestibular asymmetry among patients with a vestibular deficit less than or equal to 75% and the effectiveness of this product on acute deafferentiation syndrome and compensation among neurotomy patients.

### EXAMPLE 4

### In Vitro Effect of (D) and (L) Enantiomers of Acetyl-Leucine on Median Vestibular Neurons in the Mouse

4.1. Protocol

An electrophysiological study was conducted on mouse median vestibular neurons (MVN) in which the membrane potential was artificially maintained at various resting values. In this study, the effects of the D and L enantiomers of acetyl-leucine (1 mM) were tested on the membrane properties of neurons in “current clamp” mode at various membrane potential values: normal membrane potential (approximately −45 mV), potential maintained at a hyperpolarized level of approximately −70 mV or potential maintained at a depolarized level of approximately −35 mV.

4.2. Results

4.2.1. Effect of D and L Enantiomers of Acetyl-Leucine on Neurons Spontaneously Active at the Resting Potential

Results are presented in the following table:

| | Acetyl-L-leucine (n = 7) | | Acetyl-D-leucine (n = 8) | |
|---|---|---|---|---|
| | Before | During | Before | During |
| | application | application | application | application |
| Resting | −44.5 ± 2.1 | −42.7 ± 3.7 | −42.7 ± 3.6 | −42.0 ± 3.6 |
| potential | | (NS) | | (NS) |
| (mV) | | | | |
| Action | 14.1 ± 9.0 | 12.4 ± 7.8 | 11.5 ± 6.8 | 8.9 ± 7.7 |
| potential | | (NS) | | (NS) |
| frequency | | | | |
| (Hz) | | | | |
| Depolari- | −30.4 ± 5.6 | −27.7 ± 8.4 | −28.1 ± 4.5 | −27.0 ± 10.2 |
| zation | | (NS) | | (NS) |
| threshold | | | | |
| (mV) | | | | |
| Action | 59.0 ± 7.3 | 49.8 ± 11.4 | 59.0 ± 7.7 | 50.7 ± 17.3 |
| potential | | (p = 0.015) | | (p = 0.094) |
| height | | | | |
| (mV) | | | | |
| Membrane | 561 ± 346 | 468 ± 148 | 602 ± 58 | 644 ± 114 |
| resistance | | (NS) | | (NS) |
| (Ω) | | | | |

NS = not significant

The two enantiomers significantly decrease action potential amplitude but have no effect on other parameters.

4.2.2. Effect of D and L Enantiomers of Acetyl-Leucine on Hyperpolarized Neurons

Results are presented in the following table:

| | Acetyl-L-leucine (n = 5) | | Acetyl-D-leucine (n = 6) | |
|---|---|---|---|---|
| | Before | During | Before | During |
| | application | application | application | application |
| Membrane | −70.0 ± 2.1 | −71.6 ± 3.2 | −72.2 ± 5.2 | −67.2 ± 4.6 |
| potential | | (NS) | | (p = 0.031) |
| (mV) | | | | |
| Membrane | 364 ± 167 | 394 ± 199 | 586 ± 515 | 440 ± 364 |
| resistance | | (NS) | | (NS) |
| (Ω) | | | | |

NS = not significant

Only the D enantiomer significantly increases the membrane potential of hyperpolarized neurons.

4.2.3. Effect of D and L Enantiomers of Acetyl-Leucine on Depolarized Neurons

After several minutes of application of the D enantiomer, action potentials stop due to blocking of the membrane potential at a more depolarized level. This effect was not observed with the L enantiomer.

The results cited above show that the D enantiomer of acetyl-leucine has specific action on the membrane potential.

### EXAMPLE 5

### In Vivo Effect of the D and L Enantiomers of Acetyl-Leucine in the Rat

Preliminary tests in a surgical unilateral neurotomy model in the cat show that both D and L enantiomers of acetyl-leucine are active.

1. Use of acetyl-leucine for the preparation of a medicament to treat acute deafferentiation syndromes among patients whose vestibular deficit is less than 88%, advantageously less than 75%, to caloric tests.

2. Use according to claim 1, wherein the syndromes are intermittent acute deafferentiation syndromes resulting from an imbalance in the predominantly unilateral transmission of sensory impulses in the auditory nerve.

3. Use according to claim 1, wherein the syndromes are intermittent acute deafferentiation syndromes resulting from any event causing a unilateral loss of sensory impulse transmission in the auditory nerve, accompanied by vertigo.

4. Use according to claim 1, wherein the syndromes are acute deafferentiation syndromes resulting from surgical unilateral neurotomy.

5. Use according to claim 2, wherein the syndromes are intermittent acute deafferentiation syndromes resulting from unilateral neurotomy selected from the group comprising traumatic unilateral neurotomy, unilateral neurotomy related to acute ischemia of the auditory nerve, unilateral neurotomy due to extrinsic compression, unilateral neurotomy due to structural edemas and unilateral neurotomy resulting from endogenous viral attacks.

6. Use according to any of the preceding claims, wherein acetyl-leucine is selected from the group comprising (DL)-acetyl-leucine, (D)-acetyl-leucine and (L)-acetyl-leucine.

7. Use according to any of the preceding claims, wherein acetyl-leucine is administered by oral route or intravenous route.

8. Use according to any of the preceding claims, wherein acetyl-leucine is administered by oral route at a dose between 500 mg and 10 g per day, advantageously between 1 g and 2 g per day.

9. Use according to any of the preceding claims, wherein acetyl-leucine is administered by intravenous route at a dose of 500 mg to 1 g per day, without interruption.
